(19) United States
(12) Patent Application Publication
(10) Pub. No.: US 2025/0295355 A1
(43) Pub. Date: Sep. 25, 2025

(54) POSTURE EVALUATION DEVICE, PRIVATE BOOTH, AND POSTURE EVALUATION METHOD

(71) Applicant: Panasonic Intellectual Property Management Co., Ltd., Osaka (JP)

(72) Inventors: Kazuaki HASHIMOTO, Osaka (JP); Yosuke IZAWA, Osaka (JP); Masataka ONO, Osaka (JP); Takashi SATOU, Osaka (JP)

(21) Appl. No.: 18/861,447

(22) PCT Filed: May 9, 2023
(86) PCT No.: PCT/JP2023/017375

§ 371 Date: Oct. 29, 2024

(30) Foreign Application Priority Data
May 19, 2022 (JP) 2022-082524

(51) Int. Cl. A61B 5/00; G16H 50/30

(52) U.S. Cl. CPC A61B 5/4561; G16H 50/30

(57) ABSTRACT

A posture evaluation device (evaluation device) includes: a storage in which reference postures, which are references used in evaluation of a posture, and posture features, which correspond to the reference postures, are stored in advance; an obtainer (first obtainer) that obtains information regarding locations of body parts of a subject; a posture estimator that calculates posture features of a posture of the subject, based on the information; and an evaluator that evaluates the posture of the subject by comparing the posture features of the posture of the subject that have been calculated and the posture features corresponding to the reference postures that are stored, and outputs an evaluation result of evaluating the posture.

### TECHNICAL FIELD

The present disclosure relates to a posture evaluation device and a posture evaluation method for evaluating the posture of a subject, and to a private booth in which the posture evaluation device, or the like, is to be used.

### BACKGROUND ART

In recent years, there have been cases where the accumulation of fatigue leads to poor health, injuries, accidents, and so on. In response, attention has been given to technologies for preventing poor health, injuries, accidents, and the like, by estimating the level of fatigue. For example, Patent Literature (PTL) 1 discloses, as a fatigue estimation system for estimating a fatigue level, a fatigue determination device that determines presence or absence of fatigue and the type of the fatigue, based on force measurement and bioelectrical impedance measurement.

### CITATION LIST

### Patent Literature

[PTL 1] Japanese Unexamined Patent Application Publication No. 2017-023311

### SUMMARY OF INVENTION

### Technical Problem

However, with a conventional fatigue determination device as exemplified in the aforementioned PTL 1, there are cases where evaluation of posture from the perspective of fatigue level is not appropriate. In view of this, the present disclosure provides a posture evaluation device, and the like, that evaluates a posture more appropriately.

### Solution to Problem

A posture evaluation device according to an aspect of the present disclosure includes: a storage in which reference postures and posture features are stored in advance, the reference postures being references used in evaluation of a posture, the posture features corresponding to the reference postures; an obtainer that obtains information regarding locations of body parts of a subject; a posture estimator that calculates posture features of a posture of the subject, based on the information; and an evaluator that evaluates the posture of the subject by comparing the posture features of the posture of the subject that have been calculated and the posture features corresponding to the reference postures that are stored, and outputs an evaluation result of evaluating the posture.

A private booth according to an aspect of the present disclosure includes: the posture evaluation device described above; and partitions that define a space that accommodates the subject.

A posture evaluation method according to an aspect of the present disclosure is a posture evaluation method to be executed by a computer, and includes: storing reference postures and posture features in advance, the reference postures being references used in evaluation of a posture, the posture features corresponding to the reference postures; obtaining information regarding locations of body parts of a subject; calculating posture features of a posture of the subject, based on the information; and evaluating the posture of the subject by comparing the posture features of the posture of the subject that have been calculated and the posture features corresponding to the reference postures that are stored, and outputting an evaluation result of evaluating the posture.

### Advantageous Effects of Invention

The posture evaluation device, and the like, according to an aspect of the present disclosure can evaluate a posture more appropriately.

### BRIEF DESCRIPTION OF DRAWINGS

FIG. 1A is a first diagram for explaining the evaluation of a posture according to an embodiment.

FIG. 1B is a second diagram for explaining the evaluation of a posture according to the embodiment.

FIG. 2 is a block diagram illustrating a functional configuration of a posture evaluation system, and so on, according to the embodiment.

FIG. 3 is a diagram for explaining information stored in a storage according to the embodiment.

FIG. 4 is a diagram for explaining evaluation of a posture according to another example of the embodiment.

FIG. 5 is a diagram for explaining evaluation of a posture according to another example of the embodiment.

FIG. 6 is a diagram for explaining evaluation of a posture according to yet another example of the embodiment.

FIG. 7 is a flowchart illustrating a posture evaluation method according to the embodiment.

FIG. 8 is an external view of a private booth according to the embodiment.

FIG. 9A is a first diagram illustrating an example of a screen displayed according to the embodiment.

FIG. 9B is a second diagram illustrating an example of a screen displayed according to the embodiment.

FIG. 9C is a third diagram illustrating an example of a screen displayed according to the embodiment.

FIG. 9D is a fourth diagram illustrating an example of a screen displayed according to the embodiment.

FIG. 10 is a diagram for explaining the estimation of a posture according to a variation of the embodiment.

### DESCRIPTION OF EMBODIMENTS

Hereinafter, embodiments of the present disclosure will be described with reference to the drawings. Note that each of the embodiments described below illustrates a generic or specific example. Moreover, numerical values, shapes, materials, elements, arrangement and connection of the elements, steps, an order of steps, etc. described in the following embodiments are mere examples and are not intended to limit the present disclosure. Among elements described in the following embodiments, those not recited in any one of the independent claims are described as optional elements.

Note that the drawings are schematic and are not necessarily accurate illustrations. Moreover, elements having substantially same configurations are assigned with like reference signs in the drawings, and duplicate description may be omitted or simplified.

### Embodiment

### Configuration of System Including Posture Evaluation Device

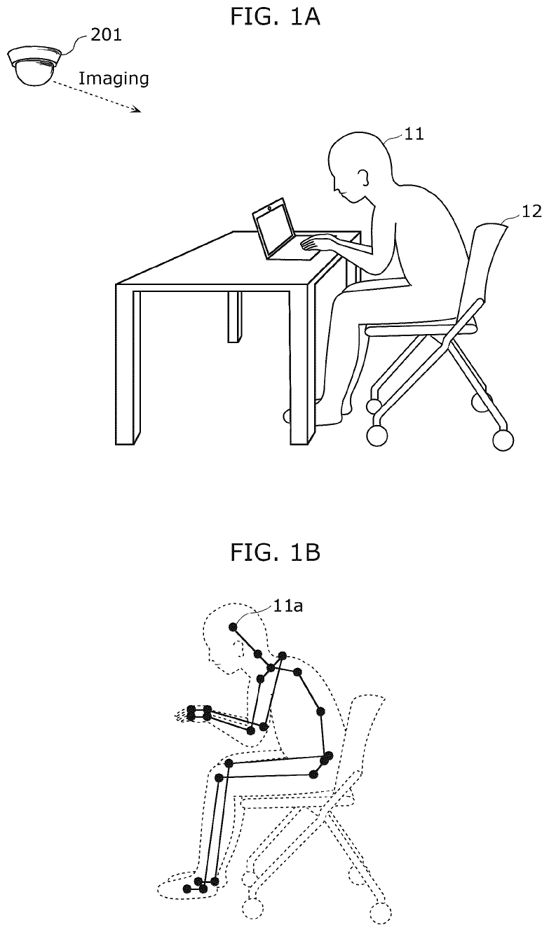

Hereinafter, an overall configuration of a posture evaluation system including a posture evaluation device according to an embodiment will be described. FIG. 1A is a first diagram for explaining the evaluation of a posture according to the embodiment. FIG. 1B is a second diagram for explaining the evaluation of a posture according to the embodiment.

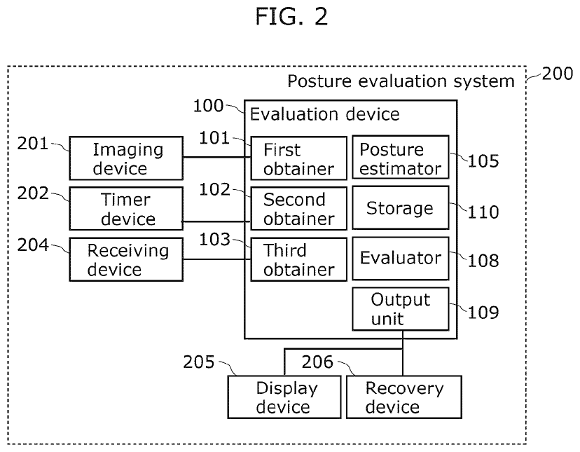

Posture evaluation system 200 (see FIG. 2 to be described later) according to the present disclosure is a system for evaluating the posture of subject 11, using images that are output by imaging device 201 after the imaging of subject 11. The form of imaging device 201 is not limited to an example described in the embodiment, and may be a fixed camera provided on a wall or in the ceiling of a building, as illustrated in FIG. 1A, or a camera provided in a PC, a smartphone, a tablet device, etc. operated by subject 11, so long as it is a camera that captures images of subject 11 and outputs the images.

The subject here is in the posture of sitting on chair 12. In posture evaluation system 200 according to the present disclosure, the posture of subject 11 is evaluated based on, among fatigue in subject 11, fatigue which is accumulated by subject 11 taking a static posture that is a fixed posture. In other words, posture evaluation is performed based on fatigue which is accumulated due to a load imposed on at least one of a muscle or a joint and a deteriorating blood flow (hereinafter also referred to as a decrease in a blood flow rate) which result from the fixed state of the posture. Accordingly, subject 11 is in the static posture of sitting, lying, or standing for at least a certain period of time. The certain period of time is a minimum period such as several seconds or several tens of seconds in which posture can be evaluated in posture evaluation system 200. Such period of time is determined depending on the processing capacities of imaging device 201 and evaluation device 100 (see FIG. 2 to be described later) included in posture evaluation system 200.

Subject 11 who takes such a static posture is, for example, a desk worker in an office, a driver who maneuvers a moving body, a person who exercises for muscle training utilizing a load imposed by a static posture, a patient in a facility such as a hospital, a passenger or a crew in an airplane, etc.

Images captured and output by imaging device 201 is processed by evaluation device 100, and the posture of subject 11 is estimated as illustrated in FIG. 1B. The estimated posture of subject 11 is output as, for example, a rigid link model 11b. Specifically, skeletal parts indicated by straight lines are connected by joints indicated by black dots, and the posture of subject 11 can be reproduced based on positional relationships between the skeletal parts and the joints, each of which is a positional relationship between two skeletal parts connected by a single joint, as illustrated in FIG. 1B. The estimation of the posture is performed through image recognition, and the estimated posture is output as a rigid link model 11b based on the positional relationships between the joints and the skeletal parts.

In the estimated rigid link model 11b, the angle formed by skeletal parts that are adjacent to each other via a joint can be calculated. Hereinafter, such an angle is referred to as a posture feature in the posture. A posture feature includes angles formed by a plurality of combinations of skeletal parts for one posture. For example, a posture feature includes the angle of the neck, the angle of the lower back, the hip joint angle, and the like. In other words, one posture is specified by the angles formed by the plurality of combinations of skeletal parts. As described above, in the present embodiment, the estimation of a posture means calculating a posture feature. With the posture feature of the estimated posture, it becomes possible to evaluate the posture corresponding to a load amount imposed on at least one of a muscle or a joint of individual body parts in order to maintain skeletal parts in the angular relationship according to the estimated posture. Here, since the load amount in at least one of a muscle and a joint of each body part is accumulated as the duration (in other words, the accumulated time during which fatigue is accumulated by maintaining the same posture) in which the above-described static posture is continued becomes longer, the fatigue level caused by subject 11 maintaining a static posture is calculated by an operation using the accumulated time. It should be noted that, in the following description, “at least one of a muscle and a joint” is also expressed as “a muscle and/or a joint.”

Furthermore, in the present embodiment, the above-described load amount imposed on a muscle and/or a joint may be read as the degree of deterioration of blood flow of subject 11.

That is, posture evaluation system 200 estimates the posture of subject 11, and thereafter evaluates, based on the accumulated time of the posture, the posture according to the fatigue accumulated in at least one perspective of the load amount on a muscle, the load amount on a joint, and the degree of deterioration of blood flow of subject 11.

It should be noted that, the higher the degree of deterioration of blood flow, the lower the blood flow rate of subject 11, and the higher the level of fatigue caused by the decreased blood flow.

Furthermore, in the subsequent evaluation of the posture of subject 11, although the evaluation of the posture is performed from the perspective of the fatigue accumulated based on at least one of the load amount on a muscle, the load amount on a joint, and the degree of deterioration of blood flow that are estimated from the posture, subjective evaluation values based on subjectivity of subject 11 generated by accumulating data such as a questionnaire are used as evaluation values (evaluation scores) associated with postures. It should be noted that, in addition to this, evaluation values may be calculated from actual measurement data of the load amount on a muscle, the load amount on a joint, and the degree of deterioration of blood flow. The actual measurement data is, namely, a database built by accumulating the measured values of the load amount on a muscle, the load amount on a joint, and the blood flow rate that have been measured for each posture in association with the posture. In posture evaluation system 200 in this case, the evaluation of the estimated posture of subject 11 can be performed by evaluation based on the measured values of the load amount on a muscle, the load amount on a joint, and the blood flow rate.

Measured data may be created by using measured values obtained for each person in consideration of differences among subjects 11 or by optimizing, for each subject 11, big data obtained from a large number of unspecified subjects through statistical analysis or analysis processing such as machine learning.

Next, a functional configuration of posture evaluation system 200 according to the present disclosure will be described with reference to FIG. 2. FIG. 2 is a block diagram illustrating a functional configuration of the posture evaluation system according to the embodiment.

As illustrated in FIG. 2, posture evaluation system 200 according to the present disclosure includes evaluation device 100, imaging device 201, timer device 202, receiving device 204, display device 205, and recovery device 206.

Evaluation device 100 is an example of a posture evaluation device and includes first obtainer 101, second obtainer 102, third obtainer 103, posture estimator 105, evaluator 108, output unit 109, and storage 110.

First obtainer 101 is a communication module that is connected to imaging device 201 and obtains, from imaging device 201, images in each of which subject 11 is captured. In other words, first obtainer 101 is an example of an obtainer. First obtainer 101 is connected to imaging device 201 by wires or wirelessly, and a method of communication performed via the connection is not specifically limited.

Second obtainer 102 is a communication module that is connected to timer device 202 and obtains a time from timer device 202. Second obtainer 102 is connected to timer device 202 by wires or wirelessly, and a method of communication performed via the connection is not specifically limited.

Third obtainer 103 is a communication module that is connected to receiving device 204 and obtains personal information from receiving device 204. Third obtainer 103 is connected to receiving device 204 by wires or wirelessly, and a method of communication performed via the connection is not specifically limited.

Posture estimator 105 is a processing unit implemented by a predetermined program being executed using a processor and memory. The posture of subject 11 is estimated through processing performed by posture estimator 105 based on images obtained by first obtainer 101. Specifically, posture estimator 105 calculates, per processing unit time, the posture feature of the posture of subject 11 at that time.

Evaluator 108 is a processing unit implemented by a predetermined program being executed using a processor and memory. Evaluator 108 estimates the posture of subject 11 based on the accumulated time of an estimated posture, using a posture estimated by posture estimator 105 and times obtained by second obtainer 102. Evaluator 108 outputs the evaluation result to output unit 109.

Output unit 109 is a communication module that is connected to display device 205 and recovery device 206 and that outputs contents that are based on the result of the evaluation of a posture performed by evaluation device 100 to display device 205 and recovery device 206. Output unit 109 is connected to display device 205 or recovery device 206 by wires or wirelessly, and a method of communication performed via the connection is not specifically limited.

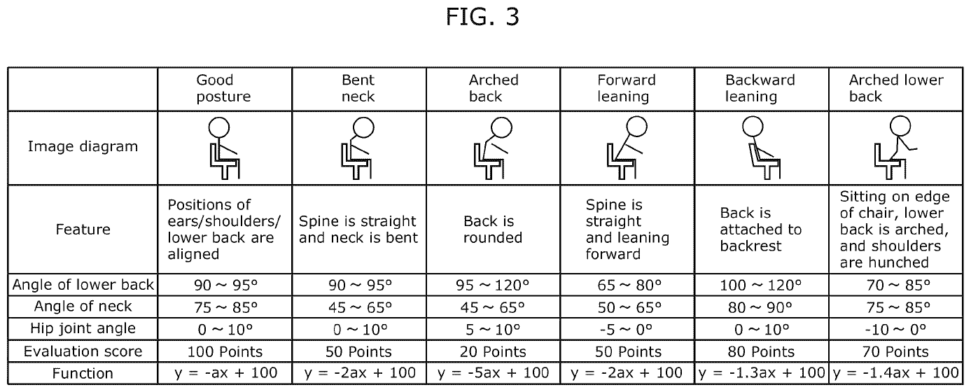

Storage 110 is a storage device that stores a plurality of reference postures that are references used in evaluation of a posture, together with posture features corresponding to the plurality of reference postures, respectively. Storage 110 stores scores together with the corresponding reference postures and posture features, such that the higher the difficulty of accumulating fatigue, the better the evaluation result, based on the fatigue level estimated for each reference posture in advance. The above-described scores may simply be numerical values, or may be the combinations of reference scores and functions that can calculate the numerical value corresponding to an accumulated time, i.e., that decreases the reference scores according to the length of the accumulated time. The scores will be described in more detail with various kinds of information stored in storage 110. FIG. 3 is a diagram for explaining information stored in the storage according to the embodiment. FIG. 3 illustrates, for each of six types of reference postures (good posture, bent neck, arched back, forward leaning, backward leaning, and arched lower back), the image diagram of the posture, the feature of the posture, the posture features (the angles of the lower back, the neck, the hip joint), the evaluation score, and the function.

In the present embodiment, the estimated posture of subject 11 is classified into any one of the plurality of reference postures according to the posture features, and the posture is evaluated from the evaluation score or the function corresponding to the reference posture. Furthermore, although not illustrated in FIG. 3, there is the other posture for classifying the posture of subject 11 when the posture does not correspond to any of the reference postures. The other posture may be set to have less influence on an evaluation result by being assigned with any of zero point, full points, or any points in between.

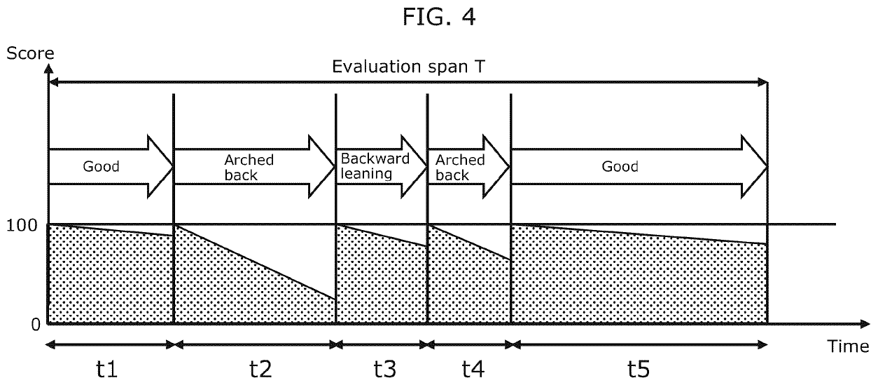

For example, the evaluation of the posture of subject 11 may be performed as follows. FIG. 4 is a diagram for explaining evaluation of a posture according to another example of the embodiment. In FIG. 4, a horizontal axis represents the period (lapsed time) during which the evaluation of the posture of certain subject 11 is being performed, and a vertical axis represents whether the posture is good or bad, which is the evaluation result. In the vertical axis, the larger the numerical value, the better the state of the posture.

In this example, the evaluation of the posture of subject 11 is performed for each evaluation span T (here, only one span is illustrated) illustrated in an upper portion of the graph. In the evaluation span in the diagram, it is illustrated that the posture of subject 11 takes, among the reference postures illustrated in FIG. 3, a good posture in a t1 period, an arched back in a t2 period, backward leaning in a t3 period, an arched back in a t4 period, and a good posture in a t5 period. As a result, in the t1 period, by using the function (y=−ax+100, where a>0) illustrated in FIG. 3, and taking the integral value of the function from 0 to t1, the evaluation value according to the accumulated fatigue can be numerically expressed, while the reference score is decreased (the evaluation of the posture is worsened, or the accumulated fatigue is increased) with a slope of −a for each unit time from 100, which is a reference score. Conversely, the accumulated value can be numerically expressed according to how little fatigue is accumulated. It should be noted that a is a numerical value determined empirically or experimentally.

Similarly, in the t2 period, by using the function (y=−2ax+100, where a>0) illustrated in FIG. 3, and taking the integral value of the function from 0 to t2, the accumulated value can be numerically expressed according to how little fatigue is accumulated, while the reference score is decreased with a slope of −2a for each unit time from 100, which is the reference score. Subsequently, the same applies to t3 to t5. In addition, the overall evaluation value of the posture corresponding to the low total accumulated fatigue can be calculated by summing the accumulated values in the evaluation span from t1 to t5. In addition, if the overall evaluation value is set to 100 in a case where the evaluation span is spent in a situation where the reference score is not decreased (there is no fatigue), it is possible to calculate what percentage the calculated actual overall evaluation value is, and output it as an evaluation result.

In this example, although the functions are set such that, each time the posture is changed, the fatigue accumulated per unit time is minimized (here, 100) by the intercept of the function according to the reference posture corresponding to the changed posture, the functions are not limited to this. For example, only the slope of a function may be specified, and a numerical value instead of the intercept may be added according to the previous progress of fatigue (the degree of decrease in the reference score). For example, in the t2 period in the diagram, the reference score is decreased to 25 at the end of t2. At this time, when it is set that 50 is added to the reference score when the posture is changed, in the t3 period, the reference score will be started to be decreased from 25+50=75. At this time, the function for the t3 period is y=−1.3ax+75. As described above, the initial value of the reference score of a subsequent period may be set according to the degree of decrease in the reference score in the previous period.

Furthermore, in this example, although the description has been given by taking the linear functions as the functions, there are cases where functions other than linear functions are suitable as functions for decreasing the reference score. Therefore, appropriate functions may be experimentally determined according to the characteristics of subject 11, or the characteristics of each reference posture.

As described above, in the present embodiment, the estimated posture of subject 11 is compared with the reference postures based on posture features to identify which of the plurality of prepared reference postures corresponds to the estimated posture of subject 11. In addition, it is possible to numerically express whether or not the posture of subject 11 is in a good state only by adding a score that is set in advance to one reference posture identified as the reference posture corresponding to the estimated posture of subject 11. That is, it becomes possible to appropriately evaluate the posture of subject 11 by relatively simple processing.

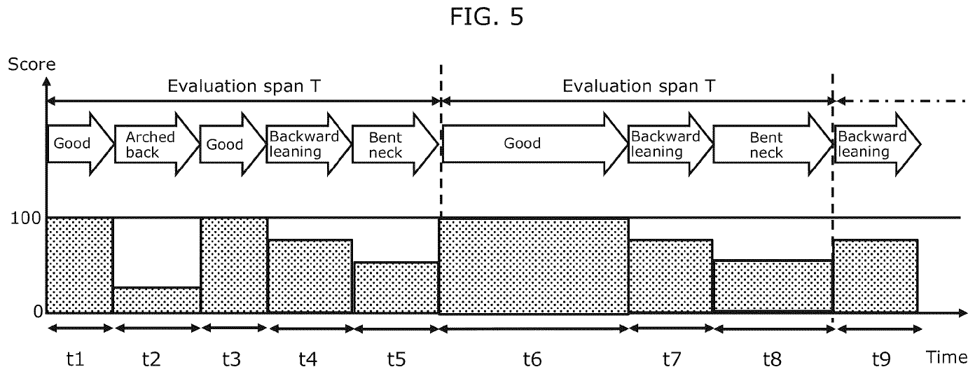

Furthermore, for example, the evaluation of the posture of subject 11 may be performed as follows. FIG. 5 is a diagram for explaining evaluation of a posture according to another example of the embodiment. In FIG. 5, a horizontal axis represents the period (lapsed time) during which the evaluation of the posture of a certain subject 11 is being performed, and a vertical axis represents whether the posture is good or bad, which is the evaluation result. In the vertical axis, the larger the numerical value, the better the state of the posture.

In a first evaluation span in the diagram, it is illustrated that the posture of subject 11 takes, among the reference postures illustrated in FIG. 3, a good posture in the t1 period, an arched back in the t2 period, a good posture in the t3 period, backward tilting in the t4 period, and bent neck in the t5 period. In this example, an example is illustrated in which the reference score is simply accumulated according to the accumulated time, without decreasing the reference score. Accordingly, in the t1 period, the accumulated value is calculated by multiplying the reference score 100 by t1 that is an accumulated time, in the t2 period, the accumulated value is calculated by multiplying the reference score 20 by t2 that is an accumulated time, in the t3 period, the accumulated value is calculated by multiplying the reference score 100 by t3 that is an accumulated time, in the t4 period, the accumulated value is calculated by multiplying the reference score 80 by t4 that is an accumulated time, and in the t5 period, the accumulated value is calculated by multiplying the reference score 50 by t5 that is an accumulated time. Thereafter, similarly, the overall evaluation value of the posture corresponding to the low total accumulated fatigue is calculated by summing the accumulated values in the evaluation span from t1 to t5, and if the overall evaluation value is set to 100 in a case where the evaluation span is spent in a situation where the reference score is not decreased, it is possible to calculate what percentage the calculated actual overall evaluation value is, and output it as an evaluation result.

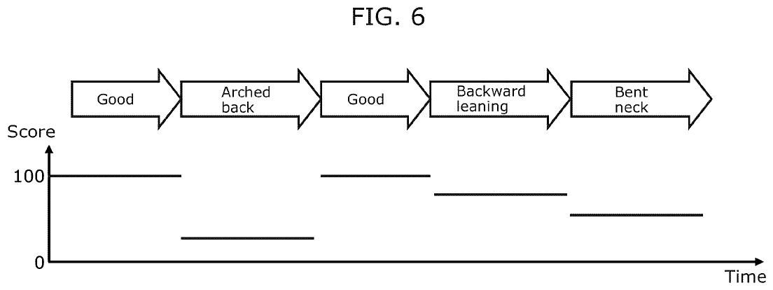

Furthermore, for example, the evaluation of the posture of subject 11 may be performed as follows. FIG. 6 is a diagram for explaining evaluation of a posture according to yet another example of the embodiment. In FIG. 6, a horizontal axis represents the period (lapsed time) during which the evaluation of the posture of certain subject 11 is being performed, and a vertical axis represents whether the posture is good or bad, which is the evaluation result. In the vertical axis, the larger the numerical value, the better the state of the posture.

In this example, an example is illustrated in which the instantaneous evaluation value of the posture is output as an evaluation result, without considering the accumulated time. Accordingly, as illustrated in the diagram, the evaluation score of 100 points is output as is during the period over which a good posture is taken, the evaluation score of 20 points is output as is during the period over which an arched back are taken, the evaluation score of 80 points is output as is during the period over which backward tilting is taken, and the evaluation score of 50 points is output as is during the period over which bent neck is taken.

As described above, the information that needs to be stored in storage 110 varies depending on how to output evaluation results. Accordingly, in addition to the reference postures and the posture features, if at least one of the evaluation scores, the reference scores, and the functions are stored in storage 110, it becomes possible to perform the evaluation of posture illustrated in one of the above-described examples.

Referring again to FIG. 2, imaging device 201 is a device that captures images of subject 11 and outputs the images, and is implemented by a camera, as described above. An existing camera such as a security camera or a fixed point camera may be used as imaging device 201 or a dedicated camera may be newly provided in a space in which posture evaluation system 200 is installed. Such imaging device 201 is an example of an information output device that outputs images as information regarding the locations of the body parts of subject 11. Accordingly, the output information is images and each of the images includes the positional relationships of the body parts of subject 11 on an imaging sensor by which subject 11 is projected.

Timer device 202 is a device that measures a time, and is implemented by a clock. Timer device 202 can send a time to second obtainer 102 to which timer device 202 is connected. The time measured by timer device 202 here may be an absolute time or an elapsed time from a relative start point. Timer device 202 may be implemented in any kind of form as long as it is possible to measure a time between two time points that are a time point at which the static state of subject 11 is detected and a time point at which the posture of subject 11 changes (i.e., accumulated time).

Receiving device 204 is a user interface that receives, as input, personal information of subject 11, and is implemented by an input device such as a touch panel or a keyboard. The personal information includes at least one of age, sex, height, weight, a muscle mass, a stress level, a proportion of fat in a body, or proficiency in performing exercise. The age of subject 11 may be a specific numerical value, or an age zone sectioned by ten years as in expressions such as teenage, twenties, and thirties, or an age zone defined by two sections with a predetermined age as a border as in an expression such as below 59 or over 60, or any other age zone.

The sex of subject 11 is an appropriate one selected out of male and female. Specific numerical values are received for the height and weight of subject 11. The compositional ratio of a muscle of subject 11 which is measured using, for instance, a body composition analyzer is received as the muscle mass of subject 11. The stress level of subject 11 is selected by subject 11 himself/herself from among, for instance, high, intermediate, and low as the subjective degree of stress felt by subject 11.

A proportion of fat in the body of subject 11 is a ratio of the weight of body fat percentage in the weight of subject 11, and is represented by, for example, percentage.

Furthermore, the proficiency of subject 11 in performing exercise may be quantified by scores attained when subject 11 performs exercise in a predetermined program, or by the conditions in which subject 11 performs exercise that subject 11 usually takes. In the former case, the proficiency is quantified by, for example, a time required for ten times of back extension, a time required for running 50 meters, or a flying distance achieved in making a long throw. In the latter case, the proficiency is quantified by, for example, how many days subject 11 performs exercise during a week or how many hours subject 11 performs exercise. It should be noted that, since personal information is used in order to improve the accuracy of evaluation of posture, such as individual optimization of evaluation scores and functions, when sufficient accuracy is secured or when generalized posture evaluation system 200 is realized, posture evaluation system 200 may be realized without including receiving device 204.

Display device 205 is a device for displaying contents that are based on the evaluation result of a posture. Display device 205 displays an image indicating the contents that are based on the evaluation result of a posture, using a display panel such as a crystal liquid panel or an electroluminescent (EL) panel. The contents displayed by display device 205 will be described later. In the case of configuring posture evaluation system 200 only to decrease the fatigue level of subject 11 by using recovery device 206 on subject 11, only recovery device 206 needs to be included in fatigue estimation system 200 and display device 205 is not essential.

Recovery device 206 is a device for decreasing the fatigue level of subject 11 by promoting the blood circulation of subject 11, for example, when, as an example, the evaluation result of the posture is unfavorable (in the case of state in which fatigue easily accumulates). Specifically, recovery device 206 performs voltage application, pressurization, vibration, or heating, or changes the arrangement of the parts of chair 12 by means of a mechanism provided in chair 12, to actively change the posture of subject 11 who is seated on chair 12. Accordingly, recovery device 206 changes the condition of subject 11 defined by at least one of a load imposed on a muscle and a load imposed on a joint, and promotes the blood circulation of subject 11. By thus promoting the blood circulation, an influence made by the deterioration of blood flow caused by subject 11 taking a static posture is reduced, and the fatigue level of subject 11 is recovered also in terms of blood circulation. Recovery device 206 is worn on or in contact with an appropriate body part of subject 11 in advance in accordance with the configuration of recovery device 206.

Note that in the case of promoting the blood circulation of subject 11 by heating, since a whole space in which subject 11 is located is heated, there is no need for subject 11 to wear or be in contact with recovery device 206 on an appropriate part of the body. In the case of configuring posture evaluation system 200 only to display the evaluation result of a posture to subject 11, only display device 205 needs to be included in fatigue estimation system 200 and recovery device 206 is not essential.

### Operation

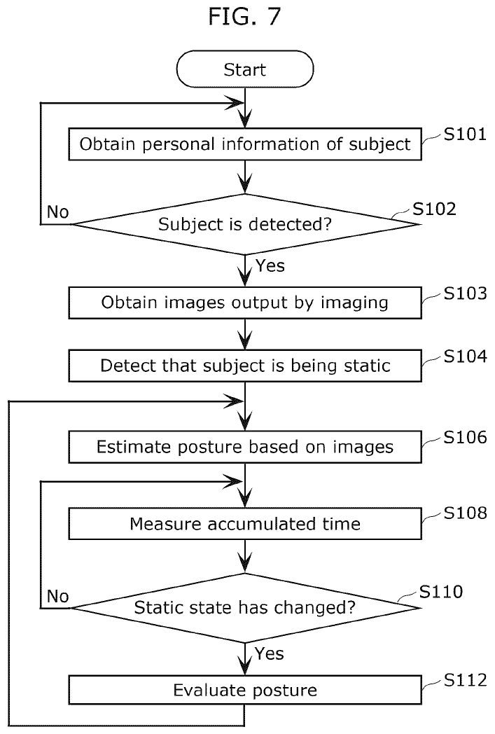

Next, the evaluation of the posture of subject 11 with the use of posture evaluation system 200 according to the embodiment will be described with reference to FIG. 7 to FIG. 9D. FIG. 7 is a flowchart illustrating a posture evaluation method according to the embodiment.

Posture evaluation system 200 first obtains personal information of subject 11 (step S101). The obtainment of the personal information is performed by input of the personal information to receiving device 204 by, for instance, subject 11 or a manager who manages the evaluation result of the posture of subject 11. The personal information of subject 11 that has been input is stored in storage 110, or the like, and is then read out and used when the posture of subject 11 is evaluated.

Posture evaluation system 200 detects subject 11, using imaging device 201 (step S102). The detection of subject 11 is performed by determining whether subject 11 appears in the angle of view of a camera that is imaging device 201. Note that subject 11 may be specific subject 11 or a person, among an unspecified number of persons, who appears in the angle of view of the camera. In the case where subject 11 is selected from the unspecified number of persons, the input of personal information may be omitted. In the case of detecting specific subject 11, a step of identifying subject 11 through image recognition or any other way is added.

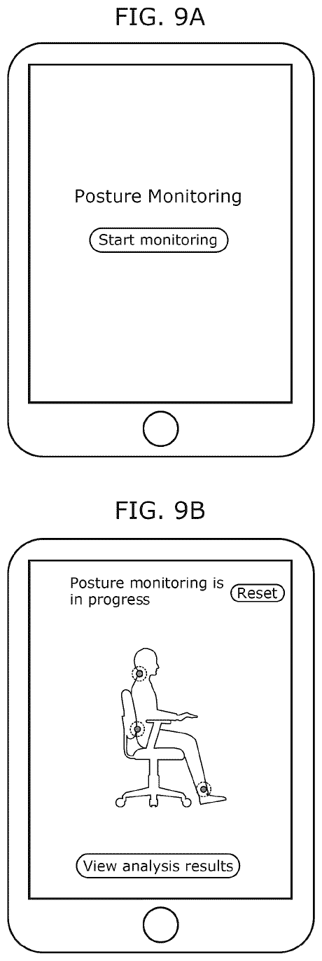

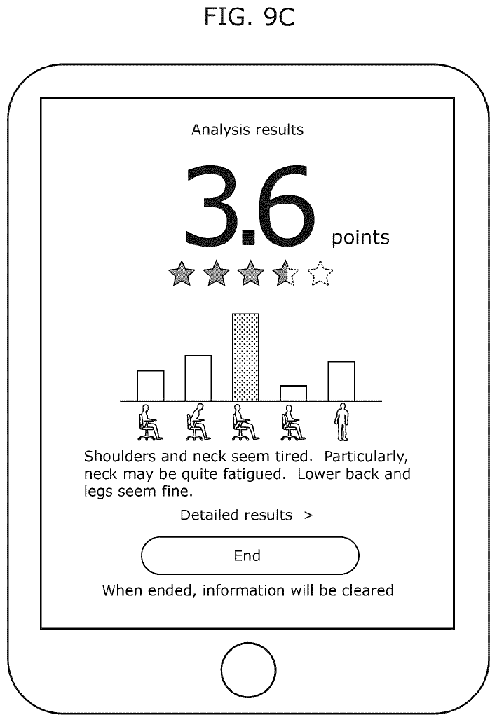

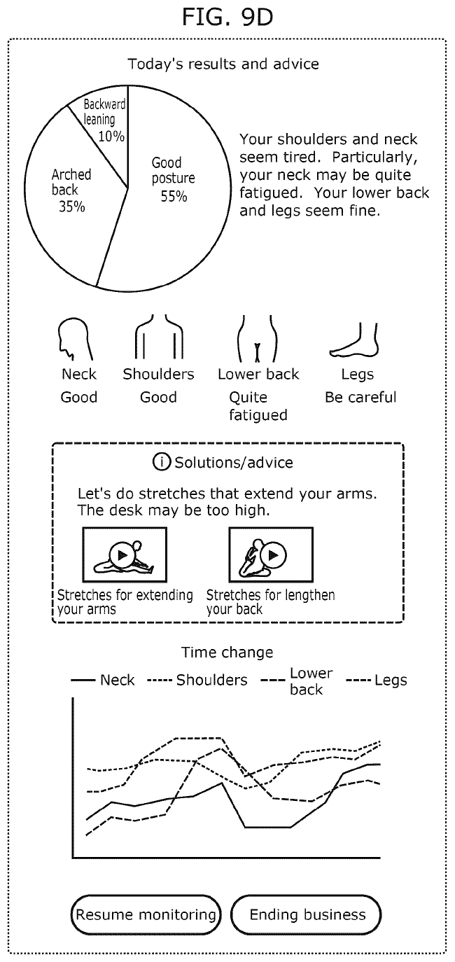

In the present embodiment, subject 11 himself/herself inputs personal information, and presses a “Start monitoring” button by, for example, operating an operation terminal (also serving as display device 205) at hand illustrated in FIG. 9A. In the operation terminal, as illustrated in FIG. 9B, a screen transitions, and it is displayed that the posture analysis is in progress (the posture monitoring is in progress), and a “View analysis results” button for displaying analysis results is also displayed. Furthermore, in an example of a screen displayed that is illustrated in FIG. 9B, a “Reset” button for discarding the monitor results of the posture accumulated so far is displayed.

In this example, the evaluation of posture is performed by identifying a detection area by imaging device 201, and entering the detection area. Particularly, here, description will be given assuming private booth 300 including posture evaluation device 100.

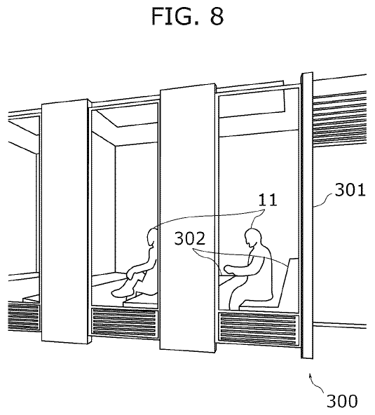

Hereinafter, private booth 300 will be described with reference to FIG. 8. FIG. 8 is an external view of a private booth according to an embodiment. As illustrated in FIG. 8, posture evaluation device 100 in this example is provided to each private booth 300 that one (in some cases, two or more) subject 11 enters, and performs work or the like in that space. Each space is separated by partitions 301 in order to define the space that can accommodate subject 11. Accordingly, since it is possible to make it easy to specify the position where subject 11 exists in the space, it becomes possible to easily optimize the arrangement of each component of posture evaluation system 200, such as imaging device 201.

Furthermore, furniture 302, such as a chair and a desk, is arranged in the spaces of private booth 300. These pieces of furniture 302 are configured to be able to be displaced according to the evaluation result of the posture of subject 11. As an example, when the evaluation result of the posture of subject 11 is relatively low, and is less than a threshold value, which requires a notification, furniture 302 can be displaced to urge changing of the posture, and the like. Furthermore, there are cases where it can be seen from the estimated posture of subject 11 that the position of furniture 302 is not appropriate. For example, when subject 11 is taking the posture of an arched back, there are cases where it can be speculated that the arrangement of a computer display for work is not appropriate. In this case, among furniture 302, the chair may be displaced in order to promote utilization of the lifting and lowering functions of the chair. Furthermore, for example, when subject 11 is taking the posture of arched lower back, there are cases where it can be speculated that the arrangement of an interface device of a computer for work is not appropriate. In this case, among furniture 302, the desk may be displaced in order to promote utilization of the lifting and lowering functions of the desk.

Referring to FIG. 8 again, in the present embodiment, since private booth 300 is allocated to each individual subject 11, image recognition or the like is not required, and the fatigue level is estimated by taking into account the personal information.

Returning to FIG. 7, first, in posture evaluation system 200, information stored in advance in storage 110 is read out. Description will be carried out under the assumption that storing of information in storage 110 is already completed.

In posture evaluation system 200, when it is determined that subject 11 is not detected (No in step S102), step S102 is repeated until subject 11 is detected. When subject 11 is detected (Yes in step S102), images that are output by imaging device 201 are obtained by first obtainer 101 (step S103, an example of obtaining information regarding the locations of the body parts of a subject in the fatigue estimation method). When subject 11 is detected as being static (being in a static posture) in the obtained images (step S104), the posture of subject 11 is estimated by estimation device 100. Specifically, posture estimator 105 estimates the posture of subject 11 based on the obtained images and pressure distribution, and calculates the posture feature (posture estimation step S106). Next, the accumulated time of the static posture of subject 11 is measured based on times obtained by second obtainer 102 (step S108). Next, whether the static state changes or not is determined by determining whether the posture estimated by posture estimator 105 changes from a certain static posture (step S110).

When it is not determined that the static state has changed (No in step S110), it returns to step S108, and continues measurement of the accumulated time.

On the other hand, when it is determined that the static state has changed (Yes in step S110), evaluator 108 evaluates the posture of subject 11 by comparing the posture feature in the calculated posture of subject 11 with the posture feature corresponding to each of the plurality of stored reference postures (step S112). Since the evaluation of posture is as described by using FIG. 4 to FIG. 6, a description here is omitted. Evaluator 108 outputs the evaluation result of the posture. Thereafter, it returns to step S106, and performs the same processing on a new posture.

Here, evaluator 108 causes storage 110 or the like to store, for example, the evaluation results (the accumulated values) for the accumulated time, and each time the evaluation span T elapses, sums the accumulated values that are output in the evaluation span, and outputs it to output unit 109 as the evaluation result. For example, subject 11 presses the “View analysis results” button by operating the operation terminal on the screen illustrated in FIG. 9B. Then, as illustrated in FIG. 9C, the numerical result indicating the evaluation result is displayed. Here, it is illustrated that the evaluation result is 72% with respect to five points in a case where there is no fatigue, that is, 3.6 points.

Furthermore, in an example of a screen displayed that is illustrated in FIG. 9C, the information on which posture subject 11 took relatively frequently (in terms of the number of times or duration) in the evaluation span is displayed by a bar graph, and the posture characteristics of subject 11 estimated from the trend is displayed. In addition, recommendation information such as advice is displayed for such posture characteristics. As described above, in the present embodiment, not only the posture is numerically evaluated, but also the posture characteristics and the like of each subject 11 can be analyzed to output beneficial information.

Furthermore, in the screen of FIG. 9C, a link for displaying a page of detailed results is included in the text “Detailed results” for displaying the evaluation result in more detail. Furthermore, an “End” button for directly ending the analysis is displayed. When the “End” button is pressed, various kinds of information obtained for evaluation of postures is deleted (cleared).

When subject 11 accesses the link “Detailed results”, transition is performed to, for example, a screen illustrated in FIG. 9D. It should be noted that FIG. 9D illustrates the entire page obtained by connecting partial pages that are displayed in parts on a screen of display device 205. When an operation (a scroll operation or the like) for displaying an arbitrary portion of this entire page is performed, the arbitrary partial page is displayed on display device 205.

In an example of a screen displayed that is illustrated in FIG. 9D, in addition to displaying the information that has been displayed in the example of the screen displayed of FIG. 9C in a different display format, the state of fatigue for each body part of subject 11 is displayed, and play buttons and the like for playing videos prepared in advance are displayed, the videos showing, as advice, what kind of actions should be specifically taken. In addition, in the example of the screen displayed of FIG. 9D, the progress of the level of fatigue accumulated for each body part of subject 11 is displayed.

### Advantageous effects, etc

As described above, evaluation device 100 (posture evaluation device) according to an first aspect of the present disclosure includes: storage 110 in which reference postures and posture features are stored in advance, the reference postures being references used in evaluation of a posture, the posture features corresponding to the reference postures; first obtainer 101 (obtainer) that obtains information regarding locations of body parts of subject 11; posture estimator 105 that calculates posture features of a posture of subject 11, based on the information; and evaluator 108 that evaluates the posture of subject 11 by comparing the posture features of the posture of subject 11 that have been calculated and the posture features corresponding to the reference postures that are stored, and outputs an evaluation result of evaluating the posture.

Evaluation device 100 as described above can evaluate the posture of subject 11 by comparing the posture of subject 11 with the reference postures stored in storage 110 in advance, and determining which of the plurality of reference postures the posture of subject 11 corresponds to. Compared with a case where the posture of subject 11 is directly evaluated with a numerical value or the like, since it is merely required to calculate which of the reference postures the posture of subject 11 corresponds to, the posture of subject 11 can be evaluated by relatively simple calculation. Therefore, the posture can be more appropriately evaluated in terms of the processing resource required for the evaluation of posture.

Furthermore, for example, evaluation device 100 according to a second aspect of the present disclosure is evaluation device 100 according to the first aspect in which, the reference postures, the posture features corresponding to the reference postures, and evaluation scores corresponding to the reference postures may be stored in advance in storage 110, and evaluator 108 may: identify, among the reference postures, a reference posture corresponding to a posture of subject 11 that has been calculated; and output, as the evaluation result, an evaluation score corresponding to the reference posture, among the evaluation scores stored.

Accordingly, the posture of subject 11 can be evaluated according to an evaluation score that is associated with each of the reference positions.

Furthermore, for example, evaluation device 100 according to a third aspect of the present disclosure is evaluation device 100 according to the first or second aspect in which, the reference postures, the posture features corresponding to the reference postures, and evaluation scores corresponding to the reference postures may be stored in advance in storage 110, and evaluator 108 may: identify, among the reference postures, a reference posture corresponding to a posture of subject 11 that has been calculated; and output, as the evaluation result, an accumulated value obtained by multiplying, among the evaluation scores, an evaluation score corresponding to the reference posture by an accumulated time indicating a period over which the posture of subject 11 is maintained.

Accordingly, the posture of subject 11 can be evaluated according to an evaluation score that is associated with each of the reference positions. At this time, it is possible to output an evaluation result that takes into account the period (accumulated time) over which the posture of subject 11 has been maintained.

Furthermore, for example, evaluation device 100 according to a fourth aspect of the present disclosure is evaluation device 100 according to any one of the first to third aspects in which, evaluator 108 may output, as the evaluation result, a total of the accumulated value per evaluation span that is a unit period over which the evaluation of the posture is performed.

Accordingly, it is possible to output a comprehensive evaluation result that takes into account several postures assumed by the subject within the evaluation span.

Furthermore, for example, evaluation device 100 according to a fifth aspect of the present disclosure is evaluation device 100 according to any one of the first to fourth aspects in which, the reference postures, the posture features corresponding to the reference postures, reference scores corresponding to the reference postures, and functions corresponding to the reference postures may be stored in advance in storage 110, the functions being for decreasing the reference scores according to a length of an accumulated time indicating a period over which the posture is maintained, and evaluator 108 may: identify, among the reference postures, a reference posture corresponding to a posture of subject 11 that has been calculated; and by using, among the reference scores and the functions, a reference score and a function that correspond to the reference posture, output, as the evaluation result, an accumulated value of the reference score for the accumulated time, the reference score having been decreased according to the function.

Accordingly, the posture of subject 11 can be evaluated according to a reference score that is associated with each of the reference positions. At this time, it is possible to output an evaluation result that takes into account the period (accumulated time) over which the posture of subject 11 has been maintained. Normally, even the same posture becomes increasingly tiring as the accumulated time becomes longer (i.e., the posture is considered bad compared to the start of the period), and thus, by decreasing the reference score according to a function, the worsening of a posture due to maintaining the same posture can be reflected in the evaluation result.

Furthermore, for example, evaluation device 100 according to a sixth aspect of the present disclosure is evaluation device 100 according to the fifth aspect in which, evaluator 108 may output, as the evaluation result, a total of the accumulated value per evaluation span that is a unit period over which the evaluation of the posture is performed.

Accordingly, it is possible to output a comprehensive evaluation result that takes into account several postures assumed by the subject within the evaluation span.

Furthermore, private booth 300 according to a seventh aspect of the present disclosure includes: evaluation device 100 according to any one of the above-described aspects; and partitions 301 that define a space that accommodates subject 11.

Accordingly, the posture of subject 11 accommodated in the space can be evaluated more appropriately.

Furthermore, for example, private booth 300 according to an eighth aspect of the present disclosure is private booth 300 according to the seventh aspect in which, furniture 302 to be used by subject 11 may be provided in the space, and a notification in accordance with the evaluation result may be performed by causing displacement of furniture 302.

Accordingly, the evaluation result of the posture of subject 11 accommodated in the space can be notified by causing displacement of furniture 302.

A posture evaluation method according to a ninth aspect of the present disclosure is a posture evaluation method to be executed by a computer, and includes: storing reference postures and posture features in advance, the reference postures being references used in evaluation of a posture, the posture features corresponding to the reference postures; obtaining information regarding locations of body parts of a subject; calculating posture features of a posture of the subject, based on the information; and evaluating the posture of the subject by comparing the posture features of the posture of the subject that have been calculated and the posture features corresponding to the reference postures that are stored, and outputting an evaluation result of evaluating the posture.

Accordingly, the same effects as those produced by evaluation device 100 described above can be produced.

### Other Embodiments

Although an embodiment of the present disclosure is described above, the present disclosure is not limited to the above embodiment.

For example, in the above embodiment, a process executed by a specific processing unit may be executed by another processing unit. An order of processes may be changed or processes may be executed in parallel.

The posture evaluation system or the evaluation device according to the present disclosure may be implemented by a plurality of devices each having one or more of the components of the posture evaluation system or the evaluation device, or by a single device having all of the components. One or more of the functions of a component may be implemented as one or more functions of another component, or each of the functions may be distributed to any of components in any way. Any form with a configuration substantially including all of the functions achievable by the posture evaluation system or the evaluation device according to the present disclosure is included in the scope of the present disclosure.

In the above embodiment, the respective components may be implemented by executing software programs suited to the respective components. The respective components may be implemented by a program execution unit such as a CPU or a processor reading and executing a software program recorded on a recording medium such as a hard disk or semiconductor memory.

The respective components may be implemented by hardware. For example, the respective components may be circuits (or integrated circuits). These circuits may compose a single circuit as a whole or may be separate circuits. Moreover, these circuits may be general-purpose or dedicated circuits.

General or specific aspects of the present disclosure may be implemented using a system, a device, a method, an integrated circuit, a computer program, or a computer-readable recording medium such as a CD-ROM, or any combination of systems, devices, methods, integrated circuits, computer programs, and recording media.

Although the posture of a subject is estimated from images, using a rigid link model generated through image recognition in the above embodiment, a method for estimating a posture is not limited to such a method. Any existing method may be used as a method for estimating the posture of a subject from images.

For example, in the above-described estimation of the posture based on images, a method of combining measurement results of a pressure sensor may be applied to the content of the present application. The pressure sensor is a sensor having a detection surface, and measures the pressure applied to each of unit detection surfaces that partition the detection surface into one or more surfaces. The pressure sensor measures the pressure for each unit detection surface in this manner, and outputs the pressure distribution on the detection surface. The pressure sensor is provided so that a subject is located on the detection surface. For example, the pressure sensor is provided to the seating surface and backrest of a chair on which a subject sits. Furthermore, for example, in the pressure sensor, a marker may be added to the detection surface, and a subject may be guided onto the detection surface by displaying “please sit on the marker” or the like. Furthermore, by guiding the subject onto the detection surface of the pressure sensor provided in a part of the floor, the pressure sensor may output the pressure distribution of the subject on the floor. For example, when uneven pressure is applied, the output pressure distribution is used for the posture estimated based on images, in order to correct the estimated posture so that the unevenness is formed.

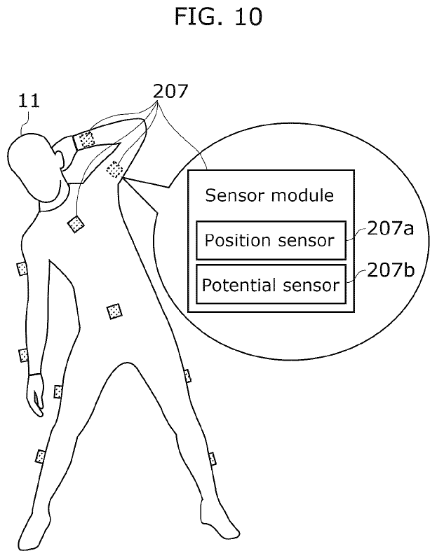

Moreover, it is also possible to implement the present disclosure by a configuration that uses a location sensor besides a configuration that uses an imaging device, as a method for estimating the posture of a subject. A specific example will be described with reference to FIG. 10. FIG. 10 is a diagram for explaining the estimation of a posture according to a variation of the embodiment. According to the present variation, the posture of subject 11 is estimated using sensor module 207 including location sensor 207a and potential sensor 207b, as illustrated in FIG. 8. Although a plurality of sensor modules 207 are worn by subject 11, the number of sensor modules 207 to be worn by subject 11 is not particularly limited. It is acceptable that only one sensor module 207 is worn by subject 11.

Moreover, how to wear sensor modules 207 is not particularly limited and any way may be allowed as long as the location of a predetermined body part of subject 11 can be measured. In FIG. 10, for example, sensor modules 207 are worn by subject 11 wearing clothing to which these sensor modules 207 are attached.

Sensor module 207 is a device worn by subject 11 on a predetermined body part and outputs information indicating the result of detection or measurement linked to the predetermined body part. Specifically, sensor module 207 includes location sensor 207a that outputs location information regarding the spatial position of the predetermined body part of subject 11, and potential sensor 207b that outputs potential information indicating an electric potential at the predetermined body part of subject 11. Although sensor module 207 including both location sensor 207a and potential sensor 207b is shown in the figure, potential sensor 207b is not essential if sensor module 207 includes location sensor 207a.

Location sensor 207a in such sensor module 207 is one example of an information output device that outputs information regarding the locations of the body parts of subject 11. Accordingly, the information to be output is location information and includes the relative or absolute location of a predetermined body part of subject 11. The information to be output may include, for example, potential information. The potential information is information including the value of an electric potential measured at a predetermined body part of subject 11. Hereinafter, the location information and the potential information will be described in detail together with location sensor 207a and potential sensor 207b.

Location sensor 207a is a detector that detects the spatial relative or absolute location of a predetermined body part of subject 11 on which sensor module 207 is worn, and outputs information regarding the spatial location of the predetermined body part as the detection result. The information regarding the spatial location includes: information that can identify the location of a body part in a space, as described above; and information that can identify a change in the location of a body part resulting from body movement. Specifically, the information regarding the spatial location includes the locations of joints and skeletal parts in a space and information indicating a change in the locations.

Location sensor 207a is composed by combining various sensors such as an acceleration sensor, an angular velocity sensor, a geomagnetic sensor, and a ranging sensor. Since the location information output by location sensor 207a can be approximated to the spatial location of a predetermined body part of subject 11, it is possible to estimate the posture of subject 11 from the spatial location of a predetermined body part.

Potential sensor 207b is a detector that measures an electric potential at a predetermined body part of subject 11 on which sensor module 207 is worn and that outputs information indicating the electric potential at the predetermined body part as the measurement result. Potential sensor 207b is measuring equipment that includes electrodes and measures a potential generated between the electrodes using an electrometer. The potential information output by the potential sensor indicates a potential generated at a predetermined body part of subject 11. Since the potential corresponds to, for instance, the active potential of a muscle at the predetermined body part, it is possible to enhance estimation accuracy in estimating the posture of subject 11 from, for instance, the active potential of the predetermined body part.

The fatigue estimation system according to the present variation estimates the fatigue level of subject 11, using the posture of subject 11 estimated as described above. Note that the processes following the estimation of the posture of subject 11 is the same as those described in the above embodiment, description is omitted.

As described above, in the fatigue estimation system according to the present variation: the information output device is location sensor 207a that is worn on a predetermined body part of subject 11 and that outputs location information regarding the spatial location of the predetermined body part as information regarding the locations of the body parts of subject 11; and evaluation device 100 estimates the posture of subject 11 based on the location information output by location sensor 207a.

Accordingly, it is possible to estimate the fatigue level of subject 11, using the location information output by location sensor 207a. In the estimation of the fatigue level of subject 11, the posture of subject 11 estimated from the output information is used. Specifically, the accumulation of fatigue due to a same static posture being kept is quantified as a fatigue level based on duration in which subject 11 keeps the static posture. Thus, in the fatigue estimation system, since the fatigue level of subject 11 is calculated in consideration of the duration of a static posture and based on the results of the detection and measurement performed by sensor module 207, it is possible to estimate the fatigue level of subject 11 in a static posture with higher accuracy and a less amount of load imposed on subject 11.

The present disclosure may be implemented as posture evaluation method executed by a posture evaluation system or an evaluation device. Moreover, the present disclosure may be implemented as a program for causing a computer to execute such a posture evaluation method, or as a non-transitory computer-readable recording medium having such a program recorded thereon.

Various modifications to the embodiments which may be conceived by those skilled in the art, as well as embodiments resulting from arbitrary combinations of elements and functions from different embodiments are included within the scope of the present disclosure so long as they do not depart from the essence of the present disclosure.

### Reference Signs List

11 subject
100 evaluation device (posture evaluation device)
101 first obtainer (obtainer)
105 posture estimator
108 evaluator
110 storage
300 private booth
301 partition
302 furniture

1. A posture evaluation device comprising:
a storage in which reference postures and posture features are stored in advance, the reference postures being references used in evaluation of a posture, the posture features corresponding to the reference postures;
an obtainer that obtains information regarding locations of body parts of a subject;
a posture estimator that calculates posture features of a posture of the subject, based on the information; and
an evaluator that evaluates the posture of the subject by comparing the posture features of the posture of the subject that have been calculated and the posture features corresponding to the reference postures that are stored, and outputs an evaluation result of evaluating the posture.

2. The posture evaluation device according to claim 1, wherein
the reference postures, the posture features corresponding to the reference postures, and evaluation scores corresponding to the reference postures are stored in advance in the storage, and
the evaluator:
identifies, among the reference postures, a reference posture corresponding to a posture of the subject that has been calculated; and
outputs, as the evaluation result, an evaluation score corresponding to the reference posture, among the evaluation scores stored.

3. The posture evaluation device according to claim 1, wherein
the reference postures, the posture features corresponding to the reference postures, and evaluation scores corresponding to the reference postures are stored in advance in the storage, and
the evaluator:
identifies, among the reference postures, a reference posture corresponding to a posture of the subject that has been calculated; and
outputs, as the evaluation result, an accumulated value obtained by multiplying, among the evaluation scores, an evaluation score corresponding to the reference posture by an accumulated time indicating a period over which the posture of the subject is maintained.

4. The posture evaluation device according to claim 3, wherein
the evaluator outputs, as the evaluation result, a total of the accumulated value per evaluation span that is a unit period over which the evaluation of the posture is performed.

5. The posture evaluation device according to claim 1, wherein
the reference postures, the posture features corresponding to the reference postures, reference scores corresponding to the reference postures, and functions corresponding to the reference postures are stored in advance in the storage, the functions being for decreasing the reference scores according to a length of an accumulated time indicating a period over which the posture is maintained, and
the evaluator:
identifies, among the reference postures, a reference posture corresponding to a posture of the subject that has been calculated; and
by using, among the reference scores and the functions, a reference score and a function that correspond to the reference posture, outputs, as the evaluation result, an accumulated value of the reference score for the accumulated time, the reference score having been decreased according to the function.

6. The posture evaluation device according to claim 5, wherein
the evaluator outputs, as the evaluation result, a total of the accumulated value per evaluation span that is a unit period over which the evaluation of the posture is performed.

7. A private booth comprising:
the posture evaluation device according to claim 1; and
partitions that define a space that accommodates the subject.

8. The private booth according to claim 7, wherein
furniture to be used by the subject are provided in the space, and
a notification in accordance with the evaluation result is performed by causing displacement of the furniture.

9. A posture evaluation method to be executed by a computer, the posture evaluation method comprising:
storing reference postures and posture features in advance, the reference postures being references used in evaluation of a posture, the posture features corresponding to the reference postures;
obtaining information regarding locations of body parts of a subject;
calculating posture features of a posture of the subject, based on the information; and
evaluating the posture of the subject by comparing the posture features of the posture of the subject that have been calculated and the posture features corresponding to the reference postures that are stored, and outputting an evaluation result of evaluating the posture.
